(19) United States
(12) Patent Application Publication
(10) Pub. No.: US 2026/0186003 A1
(43) Pub. Date: Jul. 2, 2026

(54) BLOOD ASSAYS FOR IDENTIFYING HEMORRHAGE AFTER CORONARY REVASCULARIZATION THERAPY

(71) Applicant: The Trustees of Indiana University, Bloomington, IN (US)

(72) Inventors: Rohan Dharmakumar, Zionsville, IN (US); Keyurkumar P. Vora, Bloomington, IN (US)

(73) Assignee: The Trustees of Indiana University, Bloomington, IN (US)

(21) Appl. No.: 19/133,411

(22) PCT Filed: Nov. 30, 2023
(86) PCT No.: PCT/US2023/081793

§ 371 Date: May 28, 2025

Related U.S. Application Data
(60) Provisional application No. 63/428,875, filed on Nov. 30, 2022.

(51) Int. Cl. G01N 33/68; A61K 31/16; A61K 31/4196; A61K 31/4412; A61K 31/444; A61K 31/496; A61K 45/06; G01N 33/84

(52) U.S. Cl. CPC G01N 33/6893; A61K 31/16; A61K 31/4196; A61K 31/4412; A61K 31/444; A61K 31/496; A61K 45/06; G01N 33/84; G01N 2800/324; G01N 2800/52

(57) ABSTRACT

A new blood assay is provided which identifies patients who develop hemorrhage after revascularization therapy based on temporal kinetics of free serum iron levels. This approach overcomes the limitations of gold-standard clinical practice of performing cardiac magnetic resonance imaging, as the latter is not always accessible due to poor availability and a high cost. The blood assay can be a new diagnostic approach to broadly permit classification of myocardial infarction based on hemorrhage status, and to allow for suitable management of hemorrhagic patients, who would otherwise carry a significant risk for heart failure and sudden death.

### CROSS-REFERENCE TO RELATED APPLICATIONS

This application includes a claim of priority under 35 U.S.C. § 119(e) to U.S. provisional patent application No. 63/428,875, filed Nov. 30, 2022, the entirety of which is hereby incorporated by reference.

### FIELD OF INVENTION

This invention relates to the use of a blood-based marker, serum iron levels, for identifying hemorrhagic myocardial infarction (MI) patients, especially after revascularization therapy such as reperfusion.

### BACKGROUND

In patients with acute myocardial infarction (AMI), the guideline-recommended treatment is mechanical revascularization by percutaneous coronary intervention (PCI), which is effective at reducing mortality. However, a substantial proportion of patients with AMI develop chronic cardiac failure owing to poor restoration of microvascular function and myocardial perfusion (likely as a result of microvascular obstruction), as well as irreversible microvascular injury and subsequent intramyocardial hemorrhage.

Reperfusion hemorrhage occurs in nearly 30-80% of MI patients and has recently been identified as an essential factor determining the final size of infarction. Recent studies have also shown that reperfusion hemorrhage is a key predictor of adverse outcomes in the post-infarction period. However, identifying hemorrhagic myocardial infarctions currently requires access to a cardiac magnetic resonance (CMR) imaging system, which is expensive and not readily available.

Therefore, it is an object of the present invention to provide iron-based blood assays as a new way to identify the presence and/or extent of hemorrhage in patients with myocardial infarction.

It is another object of the present invention to provide methods for improved management of hemorrhagic infarction patients following identification.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide methods for assaying blood samples of a subject with myocardial infarction or an ischemic heart disease, or of a subject in need thereof, and the methods include: detecting a first iron level and/or first ferritin level in a first blood sample obtained from the subject prior to the subject receiving a revascularization therapy, and detecting a second iron and/or second ferritin level in a second blood sample obtained from the subject after the subject receives the revascularization therapy, wherein the second blood sample is obtained within 72 hours from the revascularization therapy. In some embodiments, serum iron level is detected in the first and second blood samples. In some embodiments, plasma ferritin level is detected in the first and second blood samples. In some embodiments, serum iron level is detected in the first and second blood samples, and ferritin level is not detected from these two samples. In some embodiments, plasma ferritin level is detected in the first and second blood samples, and iron levels are not measured from these two samples. In some embodiments, both iron and ferritin levels are measured in the first and second blood samples.

In some embodiments, the second serum iron and/or second ferritin level in the second blood sample obtained at about 24 hours after the revascularization therapy is 60% or less compared to the first serum iron and/or first ferritin level, and wherein the subject has intramyocardial hemorrhage following the revascularization therapy, said hemorrhage being 5% or more in volume or area relative to the subject's myocardium.

In some embodiments, the second blood sample is obtained at about 24 hours following the revascularization therapy; optionally further comprising detecting a third serum iron and/or third ferritin level in a third blood sample obtained at about 12 hours or 48 hours following the revascularization therapy.

Typically the revascularization therapy includes but is not limited to percutaneous coronary intervention procedures, optionally being balloon angioplasty and/or stent implantation.

In some embodiments, the first blood sample is obtained within 1 hour prior to the revascularization therapy.

In some embodiments, the subject is a human. In further embodiments, the subject does not undergo cardiac magnetic resonance imaging after the revascularization therapy. In other embodiments, the subject does not undergo cardiac magnetic resonance imaging within 96 hours or within 72 hours after the revascularization therapy.

Further embodiments provide methods for treating, reducing severity, and/or inhibit hemorrhage in a subject receiving a revascularization therapy following a myocardial infarction, and the methods include: administering an iron chelator, deferrioxamine, 2,2,-Bipyridl, dexrazoxane, deferiprone, or deferasirox, to the subject, wherein the subject was detected with a lower serum iron and/or lower ferritin level in a blood sample obtained from the subject within about 72 hours after the revascularization therapy relative to respective level before the revascularization therapy, wherein the lower serum iron and/or lower ferritin level within about 72 hours after the revascularization therapy relative to respective level before the revascularization therapy indicates that the subject is likely to have developed intramyocardial hemorrhage after receiving the revascularization therapy; optionally the subject also has a lowered transferrin saturation level within the about 72 hours after the revascularization therapy relative to before the revascularization therapy.

In some embodiments, the subject was detected with the lower serum iron and/or lower ferritin level in a blood sample obtained at about 24 hours after the revascularization therapy, and the lower serum iron level and/or lower ferritin at about 24 hours after the revascularization therapy is 50% or less compared to the respective level before the revascularization therapy.

In some embodiments, the subject was detected with the lower serum iron and/or lower ferritin level in a blood sample obtained at about 24 hours after the revascularization therapy, and the lower serum iron and/or lower ferritin level at about 24 hours after the revascularization therapy is 60% or less compared to the respective level before the revascularization therapy.

In some embodiments, administering the iron chelator, deferrioxamine, 2,2,-Bipyridl, dexrazoxane, deferiprone, or deferasirox, to the subject, is within 48 hours, or within 24 hours after the revascularization therapy.

Additional embodiments provide methods for identifying a patient with myocardial hemorrhage after receiving a revascularization therapy following myocardial infarction and treating the patient, and the methods include: detecting a lower serum iron and/or lower ferritin level in a blood sample obtained from the patient within about 72 hours after the patient receives a revascularization therapy, relative to respective level of the patient before the revascularization therapy, and administering an iron chelator to the subject detected with the lower serum iron and/or lower ferritin level, based on understanding that the lower serum iron and/or lower ferritin level in a blood sample obtained within about 72 hours after a revascularization therapy relative to before the revascularization therapy indicates a presence of intramyocardial hemorrhage in the patient.

In some embodiments, the lower level is detected in the blood sample obtained at about 24 hours after the revascularization therapy, and the lower level is 60% or less compared to respective level of the subject before the revascularization therapy; and optionally the subject has a decreased transferrin saturation level at the about 24 hours after the revascularization therapy compared to before the revascularization therapy.

In some embodiments, the method further include detecting a serum iron level and/or ferritin level in a blood sample obtained from the patient at about 12 hours and/or about 48 hours after the revascularization, wherein the serum iron level and/or ferritin level at about 12 hours and/or about 48 hours after the revascularization is lower than before the revascularization, and the serum iron level and/or ferritin level at about 24 hours after the revascularization is lower than the serum iron level and/or ferritin level at about 12 hours and/or about 48 hours after the revascularization.

In various aspects, the subject or patient is indicated to have the intramyocardial hemorrhage based on the detected lower serum iron and/or lower ferritin level, and the subject or patient does not undergo cardiac magnetic resonance imaging.

Methods are also provided for diagnosing or determining presence of intramyocardial hemorrhage in a subject having received a revascularization therapy following appearance of myocardial infarction, and the methods include: detecting one or more serum iron and/or one or more ferritin levels in one or more blood samples obtained from the subject within about 72 hours after the subject receives a revascularization therapy, optionally further measuring transferrin saturation level in the blood samples, and diagnosing or determining the presence of intramyocardial hemorrhage in the subject detected with a lower serum iron and/or lower ferritin level in the one or more blood samples obtained within about 72 hours after the revascularization therapy, compared to respective level before the revascularization therapy.

In some embodiments, the detection comprises detecting a serum iron level at about 24 hours after the revascularization therapy, and the serum iron level at about 24 hours after the revascularization therapy is lower than respective level before the revascularization therapy.

In some embodiments, the one or more serum iron levels are in blood samples obtained at about 24 hours and at one or both of about 12 hours and about 48 hours after the revascularization therapy, and wherein the serum iron level at one or both of about 12 hours and about 48 hours after the revascularization is lower than before the revascularization, and the serum iron level at about 24 hours after the revascularization is lower than the serum iron level at about 12 hours and/or about 48 hours after the revascularization.

In some embodiments, one or more of the methods further include administering a therapeutic or intervention when the presence of intramyocardial hemorrhage is determined. In various aspects, the therapeutic or intervention is administered within 72 hours, 48 hours or 24 hours from determining the presence of intramyocardial hemorrhage in the subject detected based on a lower serum iron and/or lower ferritin level in the one or more blood samples obtained within 72 hours, 48 hours, or 24 hours after the revascularization therapy, compared to respective level before the revascularization therapy.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the serum iron level, normalized to baseline level, in canine MI subjects over time, before (0 hour) and after revascularization (12, 24, 48, 72 hours). Baseline level is the amount of serum iron of the MI subject before revascularization (and typically having acute myocardial infarction).

FIG. 2 depicts, in a canine model of MI, a correlation between hemorrhage volume (% LV) measured via CMR imaging and serum iron level (normalized to baseline) at 24 hours following revascularization.

FIG. 3A shows a representative human case of hemorrhagic MI. FIG. 3B shows a representative human case of non-hemorrhagic MI. Upper panels are cardiac magnetic resonance (CMR) images of representative sections of the left ventricle each after percutaneous coronary intervention (PCI) treatment; wherein the right-hand side is a late gadolinium enhancement (LGE) image, and the left-hand side is a T2* image of the same ventricular section. In these images, infarcted regions are visualized as bright hyperintensities, and hemorrhagic areas appear as dark hypodensities, providing a comprehensive and multi-modal view of the myocardial tissue changes associated with the hemorrhagic MI cases. In the lower panel, a bar graph with legends in the chart below represents iron panel data (from left to right, for each parameter) at baseline (at time of ER and before PCI), at 24 hours post-PCI, and a percentage change from the baseline to the 24 hours post-PCT. The iron panel includes parameters of serum iron, ferritin level, total iron binding capacity (TIBC), unsaturated iron-binding capacity (UIBC), and transferrin saturation. For the hemorrhagic MI case, the graph shows substantial reductions in serum iron, ferritin levels, and transferrin saturation. Conversely, the non-hemorrhagic MI case shows relatively minor changes in serum iron and ferritin levels, with noticeable shifts in TIBC and UIBC. This data offers a holistic view of hemorrhagic myocardial infarction, highlighting the contrasting diagnostic iron status alterations in the two MI scenarios and their tissue characteristics correlation with CMR findings.

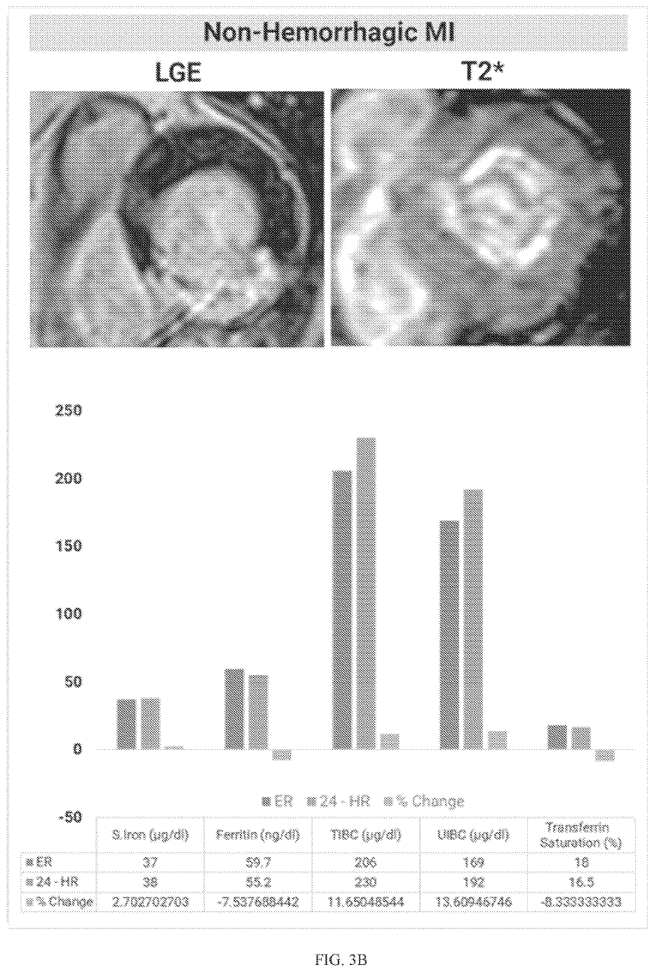

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

A “subject” means a human or animal. Usually the animal is a vertebrate such as a primate, rodent, domestic animal or game animal. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In some embodiments, a subject is a human. In other embodiments, a subject is a canine.

The term “administering,” refers to the placement an agent as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

“Revascularization” unless otherwise noted refers to myocardial revascularization herein, which may refer to a group of medical treatments that restore blood flow to parts of the heart of a subject when that flow is limited or blocked. Various embodiments provide that a patient in one or more methods disclosed herein is with ischemic heart disease, and typically isn't a candidate for other interventions such as heart bypass surgery, and hence suitable for myocardial revascularization. In some embodiments, revascularization includes coronary angioplasty, balloon angioplasty with or without stent implantation, known as percutaneous coronary intervention. In some embodiments, revascularization includes transmyocardial revascularization; whereas in other embodiments revascularization includes percutaneous myocardial revascularization.

Ferritin is a protein in cells that stores iron; and a ferritin test indirectly measures amount of iron in blood.

Transferrin Saturation (TSAT) is a percentage calculated as the serum iron concentration divided by total iron-binding capacity (TIBC).

Unless otherwise noted, the term “about” or “approximately” when used in connection with a referenced numeric indication (in percentage) means the referenced numeric indication (in percentage) plus or minus up to 5% of that referenced numeric indication (in percentage), unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

Generally acute phase of myocardial infarction refers to a period of time from the onset of coronary obstruction or symptoms of acute myocardial infarction, including amplification of reactive oxygen species activity from excess heme in the infarct zone, to the transition of ferrous to ferric iron. In some aspects, the acute phase includes the first few hours from onset of symptoms, the first few days (typically 1-3 days), and in some instances into about 1 week from onset of myocardial infarction. In some aspects, the infarct area expands in the acute phase (e.g., 48-72 hours since coronary obstruction) of myocardial infarction.

In further aspects, sub-acute phase is beyond the acute phase, and before the chronic phase. In some embodiments, the sub-acute phase is from days to weeks after MI, whereas acute phase can be hours to days after MI. Chronic phase can be weeks to months after MI. In some embodiments, an acute phase is within the first 3 days from onset of myocardial infarction symptoms, and a sub-acute phase is 7 days to 2 weeks or to 1 month since the onset of the symptoms, and a chronic phase is 3 months or later since the onset of the symptoms.

The invention relates to collecting blood samples from patients, for example when they arrive at the hospital due to a myocardial infarction and also at or throughout about 24 hours following revascularization treatment. The blood is then assayed for free iron in the serum. In various implementations, a precipitous decrease in free iron between pre-revascularization and post revascularization levels is indicative of hemorrhagic infarction. We also performed studies in canine models of infarction to validate that various extents of hemorrhagic infarctions are correlated between hemorrhage volumes identified via CMR and a decrease in serum free iron levels detected in the blood assays. Our findings indicate that the volumetric extent of the hemorrhage in the heart is indirectly proportional to the extent of decrease in pre-revascularization iron levels.

Generally, chronic phase of myocardial infarction refers to a period of weeks or months since the coronary obstruction or onset of infarction symptoms where persistent pro-inflammatory or inflammatory burden are exerted. In some aspects, the chronic phase includes 7 days and onward since the onset of infarction symptoms, e.g., 4 weeks, 8 weeks, 3 months, or 6 months.

### Sample Processing/Detection Methods

Methods are provided for assaying blood samples of a subject, wherein the subject has myocardial infarction. Methods are also provided for assaying blood samples of a subject, wherein the subject is in need of a revascularization therapy. Methods are also provided for assaying blood samples of a subject, wherein the subject has received a revascularization therapy. Methods are also provided for assaying blood samples of a subject, wherein the subject is at risk of or desires a determination of intramyocardial hemorrhage. Various provided methods for assaying blood samples include measuring serum iron level and/or ferritin level.

In various embodiments, the methods include detecting an iron (or ferritin) level in a blood sample obtained at one or more time points within about 96 hours following a revascularization therapy. In various embodiments, the methods include detecting an iron (or ferritin) level in a blood sample obtained at one or more time points within about 72 hours following a revascularization therapy. In further embodiments, the methods also include detecting an iron level in a blood sample obtained before a revascularization therapy, preferably obtained immediately before the revascularization therapy (e.g., within 30 minutes, 1 hour, 2 hours, 3 hours, 4 hours, 5 hours, or 6 hours, or within the 24 hours, prior to performing the revascularization therapy to the subject), which can be a baseline level.

Some embodiments provide that the methods detect a lower iron (or ferritin) level in the blood sample obtained within about 72 hours following a revascularization therapy, compared to the baseline level (prior to the revascularization therapy).

Some embodiments provide that the methods detect a lower iron (or ferritin) level in a blood sample obtained at about 24 hours (or between about 23-25 hours, 22-26 hours, or 21-27 hours) following a revascularization therapy, compared to the baseline level (prior to the revascularization therapy).

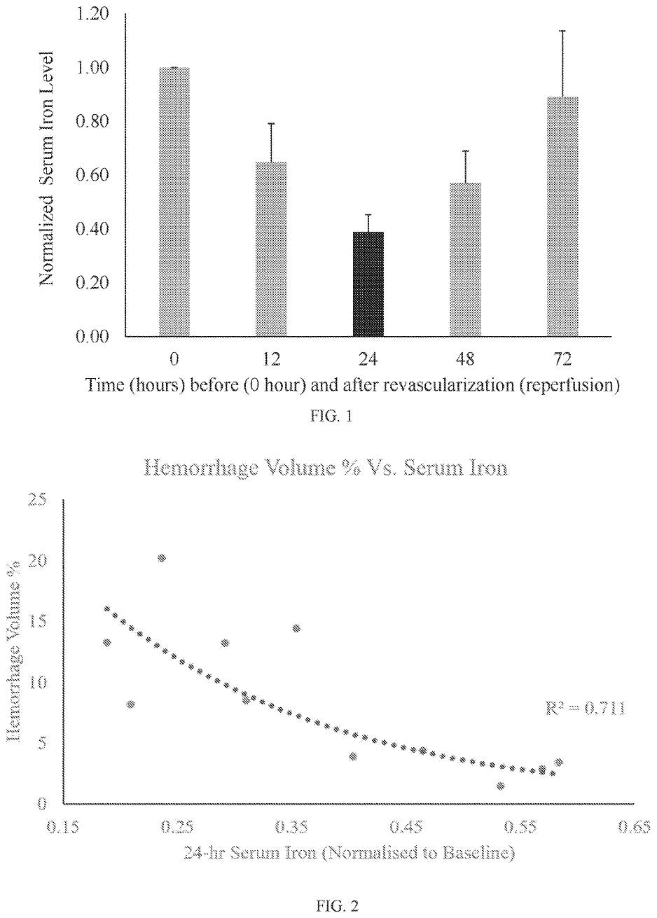

Additional embodiments provide that the methods detect decreasing levels of the iron (or ferritin) amount in a subject's blood samples over time, i.e., from his/her baseline (prior to a revascularization therapy) to about 24 hours following the revascularization therapy. Further embodiments provide that the methods detect increasing levels of the iron (or ferritin) amount in a subject's blood samples over time from about 24 hours following the revascularization therapy to about 72 hours following the revascularization therapy. Therefore, some embodiments provide the methods include first detecting decreasing levels of iron (or ferritin) in the subject's blood samples over time from prior to a revascularization therapy to about 24 hours following the revascularization therapy, and subsequently detecting increasing levels of iron (or ferritin) in the subject's blood samples over time from about 24 hours following the revascularization therapy to about 72 hours following the revascularization therapy. In several aspects, the initial “dip” (or decrease) in iron (or ferritin) levels in blood reaches a lowest amount at about 24 hours (or between about 23-25 hours, 22-26 hours, 21-27 hours, or 20-28 hours) following a revascularization therapy, which is followed by an increase in the next 1-2 days. An exemplary iron amount profile over time (kinetics) is shown in FIG. 1.

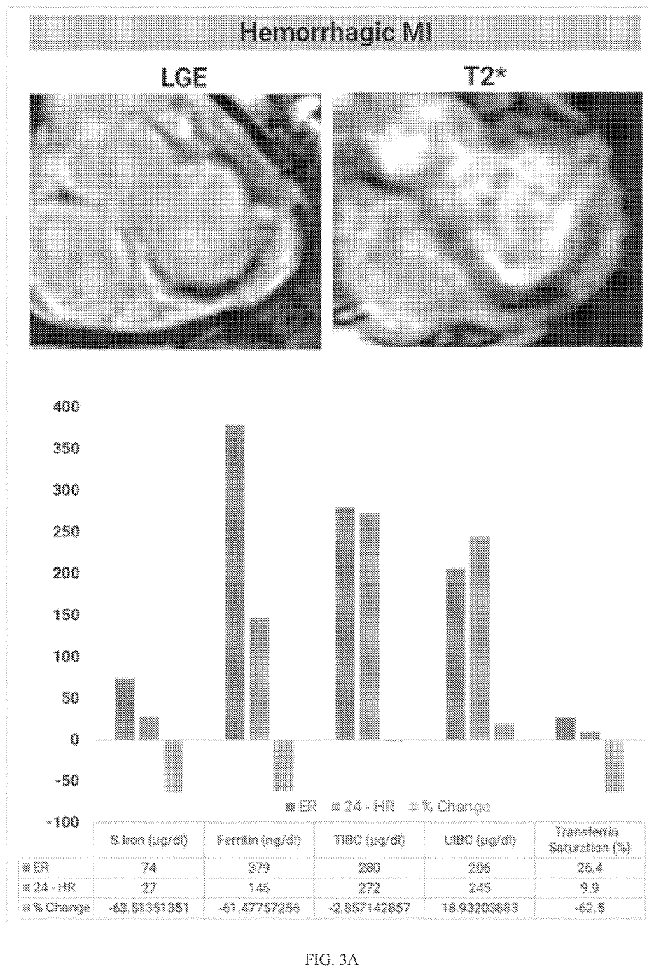

In additional aspects, the lowest amount of serum iron (or ferritin) level following the revascularization therapy, or the serum iron (or ferritin) level at about 24 hours following the revascularization therapy, is no more than 60% relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is 55% or less relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is 50% or less relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is 45% or less relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is 40% or less relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is 35% or less relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is 30% or less relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is between 10% and 20% relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is between 20% and 30% relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is between 30% and 40% relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is between 40% and 50% relative to the baseline level of the subject (prior to the revascularization therapy). In some aspects, the lowest amount of serum iron (or ferritin) level following the revascularization therapy, or the serum iron (or ferritin) level at about 24 hours following the revascularization therapy, is between 50% and 60%, relative to the baseline level. A “lowest amount” of serum iron (or ferritin) level following the revascularization can be identified as the lowest amount in three or more consecutive time points of blood samples. A lowest serum iron (or ferritin) level (following revascularization therapy such as PCI)—typically occurring at about 24 hours following the revascularization—being 60% or less, relative to the baseline level, is generally associated with the presence of an intramyocardial hemorrhage. In various instances, the lower the serum iron (or ferritin) level at about 24 hours following the revascularization, the larger the hemorrhagic volume is (as a fraction or percentage of myocardium), as validated with cardiac magnetic resonance (CMR) imaging with data shown in FIG. 2. Hence, in some implementations, a subject detected with a low serum iron (or ferritin) level at about 24 hours following revascularization being 60% or less relative to the baseline level (prior to receiving revascularization) does not need to undergo CMR imaging. In other implementations, the methods are further accompanied by performing CMR imaging of the subject to detect a presence of intramyocardial hemorrhage.

In alternative aspects, the lowest amount of serum iron (or ferritin) level following the revascularization therapy in a subject, or the serum iron (or ferritin) level at about 24 hours following the revascularization therapy, is 65% or greater relative to the baseline level of the subject (prior to the revascularization therapy); and hence the subject is indicated with an absence of intramyocardial hemorrhage, or the subject is not indicated with a presence of intramyocardial hemorrhage, or indicates that any intramyocardial hemorrhage would be below a detection limit if checked under cardiac magnetic resonance imaging.

In various aspects, the iron amount detected in one or more methods disclosed herein is iron in blood, including iron from the transferrin-iron complex (a ferric iron), its dissociated and reduced product (a ferrous iron). In additional aspects, the iron amount detected includes free ferric iron and free ferrous iron in blood, in addition to the transferrin-iron complex; especially with use of ferrozine as a chromogen to react with reduced iron. The iron amount in blood is also referred to as serum iron level.

In various aspects, a serum iron level and other serum-iron level related parameters such as total iron-binding capacity (TIBC) is measured via colorimetric techniques. For example, the Inorganic Toxicology and Nutrition Branch Division of Laboratory Sciences, National Center for Environmental Health, in Centers for Disease Control (CDC), February 2008, describes automated AAII-25 colorimetric techniques. Briefly, a serum sample may be treated with a weak acid and a reducing agent to dissociate iron from the transferrin-iron complex and reduce it from ferric to ferrous ions; and followed by measuring the intensity of colored complex formed with the ferrous ions, so as to quantify the amount of iron in the serum. Ferrous ions give a complex with Ferrozine (as chromogen). The intensity of the colored complex is proportional to the iron concentration in the sample.

In various aspects, a ferritin level is measured from blood typically drawn from a vein (e.g., from the inside of the elbow or the back of the hand) by means of electrochemiluminescence (ECLIA) and/or immunoturbidimetric techniques. For example, it can be measured in Roche Cobas e601 device with ECLIA; and it can also be measured in immunoturbidimetric Cobas c501 device. In some aspects, a measured ferritin level is a plasma ferritin level. Blood plasma can b obtained by anticoagulating the blood. Plasma ferritin levels represent the amount of ferritin in the liquid component of blood before clotting. In some aspects, serum iron is measured in blood serum, the liquid portion of blood obtained after coagulation. It represents the amount of iron circulating in the blood. In other aspects, the choice between serum and plasma measurements is user-determined and may be selected based on assay kit instructions.

In some embodiments, ferritin levels are measured via enzyme-linked immunosorbent assay (ELISA), based on the binding of specific antibodies to the target protein (ferritin). Reagents for the ELISA include anti-ferritin antibodies conjugated to an enzyme (e.g., horseradish peroxidase), substrate solution, and stop solution. For instance, a process of using ELISA to measure ferritin levels in a sample includes: adding ferritin standards and patient samples to microplate wells coated with anti-ferritin antibodies; after incubation, unbound material is washed away; then enzyme-conjugated antibodies are added, forming a complex with ferritin; and after another wash, substrate solution is added, leading to a color change; and the reaction is subsequently stopped, and the intensity of color is measured spectrophotometrically. Ferritin concentration is determined by comparing the sample's absorbance to a standard curve.

In some embodiments, TIBC and UIBC are measured in colorimetric ferrozine assay. TIBC measures the amount of iron bound to transferrin indirectly. UIBC is calculated by subtracting serum iron from TIBC. For instance, iron in a sample is released by adding a reducing agent. Released iron forms a complex with ferrozine, producing a colored compound. The intensity of color is directly proportional to the concentration of iron. TIBC is determined by adding excess iron to saturate transferrin. UIBC is calculated by subtracting serum iron from TIBC.

In some embodiments, Ferritin Saturation Percentage is calculated as: Ferritin Saturation (%)=(Serum Iron/TIBC)×100. It indicates the proportion of transferrin saturation with iron. A lower percentage may indicate iron deficiency, while a higher percentage may be seen in iron overload conditions.

In various aspects, a blood sample is fresh (assayed for quantification within 10 minutes, 20 minutes, or 30 minutes of collection, and/or without refrigeration or freezing). In some aspects, a blood sample is frozen serum. A blood sample can be harvested (or obtained or collected, used interchangeably) by standard venipuncture procedures. Therefore, in various instances, a serum iron level (or plasma ferritin level) at a certain time point refers to the serum iron level (or plasma ferritin level) in a blood sample obtained at that certain time point from a subject, although an assay to measure the iron (or ferritin) amount in the blood sample may be performed sometime later, and optionally the blood sample (e.g., optionally after serum separation) may also be frozen in between the harvest time and the assay time.

### Treatment Methods

Various embodiments provide methods for treating a subject indicated to have an intramyocardial hemorrhage, especially intramyocardial hemorrhage after receiving a revascularization therapy following myocardial infarction. Myocardial hemorrhage or intramyocardial hemorrhage may be used interchangeably herein, unless otherwise noted. The methods include administering a therapeutic agent or performing a therapy to slow down intramyocardial hemorrhage, reduce severity of hemorrhagic infarction, reduce likelihood of long-term or chronic myocardial failure, and/or improve myocardial remodeling following intramyocardial hemorrhage.

In various embodiments, exemplary therapeutic agent for this use includes but is not limited to a chelating agent (e.g., iron chelator, such as deferoxamine, deferasirox, or deferiprone), an anti-inflammatory agent, a lipid-lowering agent, a carbon monoxide therapy, a heme-oxygenase regulating drug, an agent capable of promoting heart blood flow, and a combination thereof. In various embodiments, a therapeutic agent is administered after 3 days following a revascularization therapy (or reperfusion). In some embodiments, a therapeutic agent is administered after 7 days following a revascularization therapy (or reperfusion). In some embodiments, a therapeutic agent is administered at least in a chronic phase of the myocardial infarction. Further detail of suitable therapeutic agents for treatment of intramyocardial hemorrhage or hemorrhagic myocardial infarction is described in U.S. Pat. Nos. 10,694,961 and 10,694,962 and PCT application publication no. WO2021/188984, which are incorporated by reference herein in their entireties.

In some embodiments, a method is provided for treating a subject indicated to have an intramyocardial hemorrhage after receiving a revascularization therapy following myocardial infarction, wherein the method includes administering an iron chelator to the subject, wherein the subject was detected with a lower serum iron level (or ferritin level) in a blood sample obtained from the subject within about 72 hours after the revascularization therapy relative to respective level before the revascularization therapy.

In some embodiments, a method is provided for treating a subject indicated to have an intramyocardial hemorrhage after receiving a revascularization therapy following myocardial infarction, wherein the method includes administering an effective amount of an iron chelator to the subject detected with a low serum iron level (or low ferritin level) in a blood sample harvested at about 24 hours after the revascularization therapy, said low serum iron level being 60% or less compared to respective level before the revascularization therapy. In some embodiments, a method is provided for treating a subject indicated to have an intramyocardial hemorrhage after receiving a revascularization therapy following myocardial infarction, wherein the method includes administering an effective amount of an iron chelator to the subject detected with a low serum iron level in a blood sample harvested at about 24 hours after the revascularization therapy, said low serum iron level being 60% or less compared to respective level before the revascularization therapy, and wherein the effective amount includes one or more doses and at least one of the doses is after 3 days following the revascularization therapy.

In various embodiments, methods are provided for identifying a patient with intramyocardial hemorrhage after receiving a revascularization therapy following myocardial infarction and treating the patient, wherein the methods include (1) detecting a lower serum iron level (or low ferritin level) in a blood sample obtained from the patient within about 72 hours after the patient receives a revascularization therapy, compared to respective level of the patient before the revascularization therapy, and (2) administering a therapeutic agent or performing a therapy to the subject so as to slow down the damaging effects of intramyocardial hemorrhage, reduce severity of hemorrhagic infarction, reduce likelihood of long-term or chronic myocardial failure, and/or improve myocardial remodeling, wherein the subject is detected with the lower serum iron level (or lower ferritin level), based on understanding that the lower serum iron level (or lower ferritin level) in a blood sample obtained within about 72 hours after a revascularization therapy relative to before the revascularization therapy indicates a presence of intramyocardial hemorrhage in the patient.

In various embodiments, methods are provided for identifying a patient with intramyocardial hemorrhage after receiving a revascularization therapy following myocardial infarction and treating the patient, wherein the methods include (1) detecting a low serum iron level (or low ferritin level) in a sample obtained at 24 hours or about 24 hours after the patient receives a revascularization therapy, wherein the low serum iron level (or low ferritin level) is 60% or less compared to the level before the revascularization therapy, (that is, the low serum iron level represents a 40% or more decrease from the baseline level of the patient with the intramyocardial hemorrhage but before the revascularization therapy; and (2) administering a therapeutic agent or performing a therapy to the subject so as to slow down the damaging effects of intramyocardial hemorrhage, reduce severity of hemorrhagic infarction, reduce likelihood of long-term or chronic myocardial failure, and/or improve myocardial remodeling. In some embodiments, the low iron or ferritin level at 24 hours or about 24 hours after the revascularization therapy is 50% or less compared to before the revascularization therapy in the subject, (that is, the low level represents a 50% or more decrease from the baseline level). In some embodiments, the low iron or ferritin level at 24 hours or about 24 hours after the revascularization therapy is about 40% or less than 40% compared to before the revascularization therapy in the subject, (that is, the low level represents an about 60% or more than 60% decrease from the baseline level).

In further aspects, the methods for identifying the patient with intramyocardial hemorrhage and treating the patient is based on understanding that the lower serum iron level or lower ferritin level in a blood sample obtained within about 72 hours or at about 24 hours after a revascularization therapy relative to before the revascularization therapy indicates a presence of intramyocardial hemorrhage in the patient.

In some embodiments, the methods further include detecting a serum iron level (or ferritin level) in blood samples harvested at two or more time points within 72 hours following the revascularization therapy, such as at about 12 hours, 24 hours, and/or 48 hours following the revascularization therapy. In further embodiments of the methods, a lowest amount of serum iron level (or ferritin level) is detected at about 24 hours following the revascularization therapy compared to those from samples harvested at about 12 hours and/or about 48 hours following the revascularization therapy.

### Detection and Diagnostic Methods

Various embodiments provide methods of diagnosing or determining presence of intramyocardial hemorrhage in a subject, especially in a subject having received a revascularization therapy following appearance of myocardial infarction, wherein the methods include detecting one or more serum iron levels (or one or more ferritin levels) in one or more blood samples obtained from the subject within about 72 hours after the subject receives a revascularization therapy, and diagnosing, determining, or indicating the presence of intramyocardial hemorrhage in the subject detected with a lower serum iron level (or lower ferritin level) in the one or more blood samples obtained within about 72 hours after the revascularization therapy, compared to respective level before the revascularization therapy.

In some embodiments, a method of diagnosing or determining presence of intramyocardial hemorrhage in a subject includes detecting a low serum iron level (or low ferritin level) at about 24 hours after the revascularization therapy, lowered by at least 40% compared to respective level before the revascularization therapy. That is, the serum iron level (or ferritin level) at about 24 hours after a revascularization therapy in a subject having myocardial infarction is 60% or less, compared to the respective level before the revascularization therapy, and the subject is indicated to have intramyocardial hemorrhage after the revascularization therapy. In some embodiments, a low serum iron level (or ferritin level) at about 24 hours following a revascularization therapy being about only 60% or less compared to baseline level in a human subject having had myocardial infarction, indicates that the human subject experiences intramyocardial hemorrhage following the revascularization therapy, optionally wherein the hemorrhage volume (as a fraction or percentage of myocardium) is 5% or larger.

In additional embodiments, one or more serum iron levels (or one or more ferritin levels) are measured of samples harvested at two or more time points within 72 hours following a revascularization therapy, and the serum iron level (or ferritin level) at about 12 hours and about 48 hours after the revascularization is lower than before the revascularization, and the serum iron level (or ferritin level) at about 24 hours after the revascularization is lower than respective level at about 12 hours and/or about 48 hours after the revascularization. In further aspects, the serum iron level (or ferritin level) at about 24 hours after the revascularization is 60% or less compared to before the revascularization, and the subject is indicated to have intramyocardial hemorrhage after the revascularization therapy.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Diagnostic method for identification of a type of MI after reperfusion therapy is exemplified with data shown in FIG. 1. Typically, this is a patient population with acute myocardial infarction who underwent emergency coronary angiography and revascularization by balloon angioplasty with or without stent implantation, known as percutaneous coronary intervention (PCI). Serum samples from emergency room before any PCI and 24 hours after PCI were processed to measure serum iron profile.

In an exemplary demonstration, first blood sample collected before PCI in Acute MI patients can be used as a baseline value, and a blood sample collected about 24-hour post-PCI can be a second sample. The ratio of serum iron level at 24-hour post PCI to that pre PCI can be used as a diagnostic marker to rule in or rule out hemorrhagic forms of MI (FIG. 2). Based on the current data analysis, a dip by >=40% in serum iron level at 24-hour post PCI compared to the baseline is associated with a high likelihood of hemorrhagic MI. For example, the ratio being about 0.6 or 0.5 or less indicated the presence of hemorrhage, and the smaller the ratio, the larger the hemorrhage volume percentage is (in reference to the myocardial volume).

Detailed procedures of sample preparation, reagents and equipment, and colorimetric assays followed the Lab Protocol for NHANES 2001-2002, which is described in Laboratory Procedure Manual, for detecting serum iron amount and total iron-binding capacity (TIBC) using a modified automated AAII-25 colorimetric methods, available at the Inorganic Toxicology and Nutrition Branch Division of Laboratory Sciences, National Center for Environmental Health, in Centers for Disease Control (CDC), February 2008.

Briefly, iron from the transferrin-iron complex can be dissociated by weakly acid buffer and reduced by means of reductant (Ferric→Ferrous). Ferrous ions give a complex with Ferrozine (as chromogen). The intensity of the colored complex is proportional to the iron concentration in the sample.

Serum iron was measured by a modification of the automated AAII-25 colorimetric method, which is based on the procedures of Giovaniello et al., Lab Clin Med 1968; 71:874 and of Ramsey, Clin Chem Acta 1957; 2:221. The method has been modified further to be performed on devices such as an Alpkem Flow Solutions IV (rapid-flow analysis) system. Iron is quantitated by measuring the intensity of the violet complex formed in the reaction between ferrozine and Fe⁺⁺ in acetate buffer at 562 nm. Thiourea is added to complex Cu⁺, because Cu⁺ would otherwise bind with ferrozine and yield falsely elevated iron values.

Specimens for iron analysis may be fresh or frozen serum, harvested from blood collected in a red-top (no anticoagulant) evacuated blood collection tube, by standard venipuncture procedures. A fasting sample is also preferably for better results. Specimens collected in the field are frozen and can then be shipped on dry ice by overnight mail. Serum iron is very stable, and specimens may be stored at −20 to −70° C. for years. Serum iron levels are generally not affected by freeze-thaw cycles. Hemolyzed specimens should not be used because of the contribution of iron from hemoglobin. In general, plasma should be transported and stored at no more than −20° C. Samples thawed and refrozen less than five times are not compromised. Specimens were allowed to gradually reach room temperature before sample preparation and during testing.

Standards can be prepared by:

### (1) g/L Stock Iron Standard Solution

Place 1.000 g iron wire in a 1-L volumetric flask. Add 12 mL concentrated HCl and dissolve the wire with slight warming. After the wire is completely dissolved, cool the flask to room temperature and dilute contents to volume with deionized water. (The solution is stable indefinitely; store in a polypropylene container at 20-25° C.)

(2) 50.0 mg/L Iron Intermediate Stock Solution

Dilute 25 mL of the 1.0 g/L stock iron solution to 500 mL with 0.1 mol/L HCl. (Prepare each time new working standards are required.)

### (3) Working Iron Standards

In a series of 500-mL volumetric flasks, prepare dilutions from the intermediate standard as shown below. Dilute to 500 mL with 0.1 mol/L HCl and mix well. (Prepare as needed; the solution is stable at 20-25° C.)

### (4) NIST Iron Standard (SRM 937)

This standard reference material has a concentration of 1.0001 mg/mL and may be diluted at concentrations from 1 to 1000 μL/dL as a verification of the accuracy of the working standard dilutions.

The serum iron concentration of specimens is calculated from the slope and y intercept of the >=8-point regression line of the expected concentrations of the standards versus their millivolt recorder values (or absorbance values). R² for the regression line should be >0.9990. The method is linear from 0 to 1000 μL/dL. Specimens with iron values<30 μL/dL or >200 μL/dL are reanalyzed for confirmation. The limit of detection is approximately 2.0 μL/dL.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

An additional example includes but is not limited to BECKMAN COULTER® analyzer BC Serum Iron Protocol BAOSR6x86.01, 2009-08, incorporated herein by reference in its entirety.

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method for assaying blood samples of a subject with myocardial infarction or an ischemic heart disease, comprising:
detecting a first serum iron and/or first ferritin level in a first blood sample obtained from the subject prior to the subject receiving a revascularization therapy, and
detecting a second serum iron and/or second ferritin level in a second blood sample obtained from the subject after the subject receives the revascularization therapy,
wherein the second blood sample is obtained within 72 hours from the revascularization therapy.

2. The method of claim 1, wherein the second blood sample is obtained at about 24 hours after the revascularization therapy, and the second serum iron and/or second ferritin level is 60% or less compared to the first serum iron and/or first ferritin level, and wherein the subject has intramyocardial hemorrhage following the revascularization therapy, said hemorrhage being 5% or more in volume or area relative to the subject's myocardium.

3. The method of claim 1, wherein the second blood sample is obtained at about 24 hours following the revascularization therapy, and the method further comprises detecting a third serum iron and/or third ferritin level in a third blood sample obtained at about 12 hours or 48 hours following the revascularization therapy.

4. The method of claim 1, wherein the revascularization therapy comprises percutaneous coronary intervention procedures, optionally being balloon angioplasty and/or stent implantation.

5. The method of claim 4, wherein the first blood sample is obtained within 1 hour prior to the revascularization therapy.

6. The method of claim 1, wherein the subject is a human.

7. The method of claim 1, wherein the subject does not undergo cardiac magnetic resonance imaging after the revascularization therapy.

8. The method of claim 7, wherein the subject does not undergo cardiac magnetic resonance imaging within 96 hours or within 72 hours after the revascularization therapy.

9. A method for treating, reducing severity, and/or inhibit hemorrhage in a subject receiving a revascularization therapy following a myocardial infarction, the method comprising:
administering an iron chelator, deferrioxamine, 2,2,-Bipyridl, dexrazoxane, deferiprone, or deferasirox, to the subject, wherein the subject was detected with a lower serum iron and/or lower ferritin level in a blood sample obtained from the subject within about 72 hours after the revascularization therapy relative to respective level before the revascularization therapy,
wherein the lower serum iron and/or lower ferritin level within about 72 hours after the revascularization therapy relative to respective level before the revascularization therapy indicates that the subject is likely to have developed intramyocardial hemorrhage after receiving the revascularization therapy; optionally the subject also has a lowered transferrin saturation level within the about 72 hours after the revascularization therapy relative to before the revascularization therapy.

10. The method of claim 9, wherein the subject was detected with the lower serum iron and/or lower ferritin level in a blood sample obtained at about 24 hours after the revascularization therapy, and the lower serum iron level and/or lower ferritin at about 24 hours after the revascularization therapy is 50% or less compared to the respective level before the revascularization therapy.

11. The method of claim 9, wherein the subject was detected with the lower serum iron and/or lower ferritin level in a blood sample obtained at about 24 hours after the revascularization therapy, and the lower serum iron and/or lower ferritin level at about 24 hours after the revascularization therapy is 60% or less compared to the respective level before the revascularization therapy.

12. The method of claim 9, wherein administering the iron chelator, deferrioxamine, 2,2,-Bipyridl, dexrazoxane, deferiprone, or deferasirox, to the subject, is within 48 hours, or within 24 hours after the revascularization therapy.

13. A method for identifying a patient with myocardial hemorrhage after receiving a revascularization therapy following myocardial infarction and treating the patient, comprising:
detecting a lower serum iron and/or lower ferritin level in a blood sample obtained from the patient within about 72 hours after the patient receives a revascularization therapy, relative to respective level of the patient before the revascularization therapy, and
administering an iron chelator to the patient detected with the lower serum iron and/or lower ferritin level, based on understanding that the lower serum iron and/or lower ferritin level in a blood sample obtained within about 72 hours after a revascularization therapy relative to before the revascularization therapy indicates a presence of intramyocardial hemorrhage in the patient.

14. The method of claim 13, wherein the lower level is detected in the blood sample obtained at about 24 hours after the revascularization therapy, and the lower level is 60% or less compared to respective level of the patient before the revascularization therapy; and optionally the patient has a decreased transferrin saturation level at the about 24 hours after the revascularization therapy compared to before the revascularization therapy.

15. The method of claim 14, further comprising detecting a serum iron level and/or ferritin level in a blood sample obtained from the patient at about 12 hours and/or about 48 hours after the revascularization, wherein the serum iron level and/or ferritin level at about 12 hours and/or about 48 hours after the revascularization is lower than before the revascularization, and the serum iron level and/or ferritin level at about 24 hours after the revascularization is lower than the serum iron level and/or ferritin level at about 12 hours and/or about 48 hours after the revascularization.

16. The method of claim 9, wherein the patient is a human.

17. The method of claim 9, wherein the patient is indicated to have the intramyocardial hemorrhage based on the detected lower serum iron and/or lower ferritin level, and the patient does not undergo cardiac magnetic resonance imaging.

18. (canceled)

19. (canceled)

20. The method of claim 13, wherein the detecting step comprises detecting lower serum iron levels in blood samples obtained at about 24 hours after the revascularization therapy and at one or both of about 12 hours and about 48 hours after the revascularization therapy, each being lower relative to before the revascularization therapy; and wherein the serum iron level at the about 24 hours after the revascularization is lower than the serum iron level at the about 12 hours and/or the about 48 hours after the revascularization.

21. The method of claim 2, further comprising administering a therapeutic or intervention to the subject.

22. The method of claim 3, further comprising administering a therapeutic or intervention to the subject within 72 hours, 48 hours or 24 hours from detecting a lower serum iron and/or lower ferritin level in the second or the third blood sample compared to the first blood sample.
